(19) United States
(12) Patent Application Publication
(10) Pub. No.: US 2023/0277863 A1
(43) Pub. Date: Sep. 7, 2023

(54) Use Of Photosensitizers For Treatment Of Viral Respiratory Infections

(71) Applicants: Jochen Arentz, Hamburg (DE); Hans Albrecht, München (DE)

(72) Inventors: Jochen Arentz, Hamburg (DE); Hans Albrecht, München (DE)

(21) Appl. No.: 18/017,602

(22) PCT Filed: Jul. 22, 2021
(86) PCT No.: PCT/EP2021/070514

§ 371 Date: Jan. 23, 2023

(30) Foreign Application Priority Data
Jul. 22, 2020 (EP) 20187269.4

(51) Int. Cl. A61N 5/06; A61K 9/00; A61K 41/00; A61N 5/067; A61P 31/14

(52) U.S. Cl. CPC A61N 5/0603; A61K 9/0078; A61K 41/0057; A61N 5/067; A61N 5/062; A61P 31/14; A61N 2005/0652; A61N 2005/0663; A61N 2005/0644; A61N 2005/0604

(57) ABSTRACT

Treatment of respiratory viral infections including coronavirus using photosensitizers. A photoactivatable phenothiazine derivative, preferably a photoactivatable phenothiazine dye such as methylene blue, new methylene blue, toluidine blue and mixtures thereof is administered to the naso-pharyngeal tract of a subject, and is then activated with a light source.

### FIELD OF THE INVENTION

The present invention relates to the treatment of viral infections using photosensitizers. More particularly, the present invention provides treatments for infections by viruses infecting the respiratory tract of a subject, in particular humans. The present invention also relates to pharmaceutical devices comprising means for application of a suitable photosensitizer in combination with nebulizer so as to administer the photosensitizer into at least a part of the respiratory tract of a subject to be treated. The invention furthermore relates to kits for use in the inventive treatments comprising a device of the invention and suitable light source.

### BACKGROUND OF THE INVENTION

Photosensitizers have been used for photodynamic therapy (PDT) of infections by bacteria and fungi as well as cancer and tumours. Photosensitizers are generally dyes which are transferred to a higher energetically state by absorption of light typically in the visible wavelength range. In the photo-activated state the photosensitizer is able to transfer, as a donor, at least part of the energy to an acceptor molecule. For application in PDT, the energy transfer leads, at least in part, to reactive oxygen species (ROS) such as the superoxide radical which in turn damages various cellular components, especially the cell membrane, of the target cells such as tumour cells, bacteria, or fungal cells, by redox reactions. PDT is mostly applied through local application of the dye and light typically emitted by monochromatic and/or coherent light sources.

### SUMMARY OF THE INVENTION

The technical problem underlying the present invention is to provide improved treatment of viral infections in the respiratory tract as well as novel therapeutic devices and kits for performing the treatment.

In particular, the present invention provides, under a first aspect, the use of at least one photosensitizer in the treatment of viral respiratory infections.

In order to provide the photosensitizer at the required amount and concentration at the region of the targeted viruses, it is preferred that the photosensitizer is applied inra-nasally and/or intra-orally and/or intra-pharyngeally.

As mentioned above, the photosensitizer needs to be activated in order to exert its function of at least damaging or, optimally, eliminating viruses infecting the respiratory tract of the subject to be treated, which is accomplished by light emission on the part or region of the subject in need of the inventive treatment where the photosensitizer has been applied to. Typically, the light emission is directed to the respiratory tract, or at least a part thereof, of the subject. The light for irradiating at least a part of the respiratory tract has a wavelength, or at least comprises a wavelength, and typically also an intensity, preferably in terms of energy density, i.e. applied energy per surface area, causing the photosensitizer to be transferred to an active state as outlined above.

In certain embodiments of the invention the treatment comprises irradiating at least a part of the respiratory tract of a subject from outside of the subject with light comprising or having a wavelength causing the photosensitizer to be transferred to an active state. It is conceived that one or more different parts of the respiratory tract may be irradiated one or more times for a time effective to provide the sufficient light energy so as to effect the inventive transfer of the photosensitizer, in particular the effective amount administered to the respiratory tract, or one or more parts thereof, to the active state at least in the irradiated part or region, or whole of, respectively, of the respiratory tract. Parts of the respiratory tract which may be conveniently irradiated from the outside of the subjects body include, e.g. nose, mouth (preferably, the subject's mouth can be irradiated while in the opened or at least partially opened state), cheeks (one side or both sides, preferably left and right cheek simultaneously or sequentially), neck, throat, chest and/or back of the subject to be treated. It is to be understood that the term “from the outside of the subject's body” means that the irradiation is directed to the skin of the subject in the respective area, in the following also denoted as “transcutaneous irradiation”, with two exceptions, namely, the mouth and the nose: as outlined before, the mouth of the subject can be irradiated in an open state where the emitted light will also, or mainly, irradiate the oro-pharyngel part of the respiratory tract of the treated subject. The nose of the subject can also be treated by emitting light into the nostrils of the patient so that the light also, or mainly, reaches the nasal part of the subject's respiratory tract. In any case, however, irradiation from the outside of the subject's body means that the light-emitting device is not introduced into any parts of the respiratory tract.

In other preferred embodiments of the invention at least a part of the respiratory tract of the subject in the need of the inventive treatment is irradiated within the subject's body, preferably by using a light source which is introduced into the infected or at least suspected to be infected subject. In an embodiment thereof, the light source is equipped with fibre optical elements, e.g. a waveguide, dimensioned, in particular with respect to its diameter and length, to be introduced intra-nasally and/or intra-orally into the subject's respiratory tract and pushed to the desired site of light emittance or irradiation, respectively. The irradiation of the subject from inside the body, preferably via suitable fibre optics, can be directed to one or more desired parts of the inner respiratory tract such as intra-nasal, intra-oral, nasopharyngeal, pharyngeal and/or tracheal irradiation. Fibre optics suitable for use in the invention are generally known in the art and commercially available, for example from CeramOptec GmbH, Bonn, Germany.

In certain preferred embodiments of the invention, a combined irradiation treatment is performed wherein irradiation of the nostril mucosa is carried out, preferably in both nostrils, using intra-nasal irradiation (i.e. irradiation is carried out inside the patient's body) and, simultaneously or, preferably, sequentially, the mucosa of the mouth and/or throat is irradiated from outside of the patient's body.

The light source used for irradiation according to the invention, which, according to the invention, may refer to light emitting element of a corresponding device or the device per se, is preferably a device comprising a light emitting diode (LED). For convenience of use, the LED device is, particularly when for use from outside of the subject's body, a handheld light emitting device, more preferably a handheld LED device such as a device having the form and comprising the typical parts of, respectively, a torch or flashlight, respectively. In embodiments of the invention the light source emits coherent light, in which case the light source is a laser device emitting light of a specific wavelength. The laser device can be an LED laser device, and in certain embodiments, it may be a handheld laser LED device, as outlined above. In other preferred embodiments, the light source or light emitting device, respectively, can be a light source or device, respectively, emitting non-coherent light, preferably an LED device, more preferably a handheld LED device. It is a surprising finding according to the invention that it is sufficient for attaining substantial and strong virus inhibition to use a broadband light source, more preferably a broadband LED device, more preferably a broadband handheld LED device, preferably emitting white light, most preferred warm white light. In preferred embodiments of broadband LED devices, the LED light source is doted with Cer which shifts the potential dominant blue part (shorter wavelength) of the emitted light to longer wavelengths. In other preferred embodiments the LED devices can be one emitting non-coherent monochromatic light, preferably having a wavelength as further described below.

In certain preferred embodiments of the invention, a combined irradiation inside and from outside of the patient's body can be attained using the same light source, but with different additional elements for irradiation inside the patient's body and for irradiation outside of the human body. In one preferred exemplary embodiment of this type, a handheld light emitting device, preferably an LED device, more preferred a handheld broadband LED device, most preferred a torch-type broadband LED device, can be equipped for inside irradiation, preferably intra-nasal irradiation, with a removably affixable light-guiding element. Preferably, a light-guiding element has a portion for removable fixation of the element to the, preferably handheld, LED device in the region, preferably an end of the LED device, where the emitted light exits the light emitting device, in case of a torch-type device an upper or (from the perspective of the user holding the torch-type device at the proximal part thereof) distal end thereof, such as a part or member forming a circumferential ring or partly circumferential part sized to fit the diameter of the light-emitting, preferably top or distal, part of the LED device, and a portion, i.e. a light guiding portion, having a conical shape extending from the fixation part, preferably having the form of the top conus of an intra-nasal pump spray device, such that the light guiding portion can be introduced into the subject's body, preferably into a nostril of the subject. For irradiation from outside the subject's body, the light emitting device can be equipped with a removably affixable element for positioning the device at a desired location of the patient's body where the irradiation from outside the patient's body is to take place. Preferably, a positioning element comprises a fixation portion, more preferred a fixation portion as described for the inside irradiation element, and an attachment portion for, preferably gently, attaching the light-emitting device to the desired portion of the patient's body. In a preferred embodiment, the attachment portion comprises or consist of, respectively, a part sized to be positioned against a part of the subject's body such as the upper lip in the case of irradiating the mouth and/or throat of the subject to be treated. In preferred embodiments, the (preferably removably) affixable elements are devised for single or multiple use such as made of a disposable material for single or multiple use. In other embodiments, the removably affixable elements may be sterilized after one or more such as two, three or four uses. In preferred embodiments of the invention, a light emitting device, preferably a handheld device, more preferably a broadband LED device and removably affixable elements for inside and/or outside irradiation, preferably elements as described above, can be included in a kit, preferably together with a container comprising the photosensitizer, preferably a solution thereof, such as, in highly preferred embodiments, a nasal spray device.

Preferred photosensitizers for use in the invention are photoactivatable phenothiazine derivatives, more preferably photoactivatable phenothiazine dyes. Phenothiazine photosensitizers for use in the invention typically have a structure according to the following general formula (I):

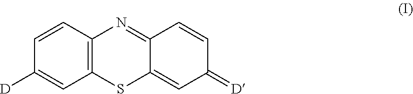

with D and D′ each being an electron donor group, and wherein the positions C-1, C-2, C-4, C-6, C-8 and/or C-9 of the phenothiazine ring system may be substituted by a substituent preferably selected from substituted or unsubstituted lower alkyl, preferably C₁₋₃-alkyl, particularly preferred methyl, or lower alkenyl, preferably C₂₋₃-alkenyl, amino, halo, and cyano. Preferably, D and D′ are each independently a NRR′ group with R and R′ being preferably independently selected from H, substituted or unsubstituted lower alkyl, preferably C₁₋₃-alkyl, particularly preferred methyl, and lower alkenyl, preferably C₂₋₃-alkenyl.

Particularly preferred phenothiazine derivatives for use in the invention are methylene blue, new methylene blue, toluidine blue and mixtures thereof.

The photosensitizer for use in the invention, preferably a phenothiazine derivative as described above, is typically applied in form of a composition containing at least one photosensitizer such as a photoactivatable phenothiazine in combination with at least one excipient and/or diluent, preferably at least one pharmaceutically acceptable excipient and/or diluent. Preferred diluents/excipients for use in the invention are typically water, preferably sterilized water, more preferably sterilized distilled water, or aqueous solutions such saline or buffered saline solutions such as PBS, whereby it is understood that such diluents and/or excipients are preferably sterilized.

It has been surprisingly found according to the invention that photosensitizers as taught herein, preferably phenothiazine derivatives of above formula (I), particularly preferred methylene blue, new methylene blue, toluidine blue or mixtures thereof, can be applied in comparatively very low concentrations for effectively combating viruses causing respiratory conditions. In preferred embodiments the composition comprises a photosensitizer for use in the invention, particularly preferred a phenothiazine derivative as defined herein, in a concentration of from about 0.00001% (w/v) to about 0.1% (w/v), preferably from about 0,0001% (w/v) to about 0,001% (w/v).

The photosensitizer, preferably contained in a composition as outlined above, is preferably administered to the respiratory tract of the subject through nasal and/or oral application. For example, the photosensitizer, in particular the composition containing at least one photosensitizer (i.e. one or more of such photosensitizers) as outlined above may be administered via a pipette or similar device. For administration to deeper parts of the respiratory tract tube systems known in the art equipped with a syringe reservoir at its proximal part can be used. In preferred embodiments, allowing for reaching the upper and lower parts of the respiratory tract of the subject to be treated, the photosensitizer(s) is (are) preferably administered by use a device configured to administer the photosensitizer(s), preferably a composition containing the photosensitizer(s), in spray or nebulized from. Typically, such a device comprises a nebulizer element and a container comprising a composition containing one or more photosensitizers as described above.

The invention is also directed to such medical devices for administration of the photosensitizers in spray or nebulized, respectively, form. In particular, the present invention provides a pharmaceutical device comprising a nebulizer and a container containing at least one photosensitizer, optionally in combination with at least one pharmaceutically acceptable excipient and/or diluent, wherein preferred embodiments it referred to the have been already described in detail herein-above.

As already noted above, the wavelength or wavelength spectrum of the light emitted by the light source depends on the particular photosensitizer. Preferred phenothiazine photosensitizers for use in the invention typically absorb light in the visible to near infra red light of the electromagnetic wave spectrum. For example, in the visible spectrum, methylene blue absorbs light in the wavelength region of from about 530 nm to about 700 nm, new methylene absorbs light in the wavelength region of from about 500 nm to about 700 nm, and toluidine blue absorbs light in the region of from about 520 nm to about 700 nm. In preferred embodiments, the photosensitizer is typically activated by irradiation with light have a wavelength of wavelengths, respectively, in the range of from about 500 nm to about 820 nm. As already indicated above, the light source or light emitting device, respectively, for use in the invention emits light typically within this wavelength region, whereby the light source can emit light in broadband region, preferably, of the above indicated visible to near infrared spectrum, preferably of about 500 nm to about 820 nm. Particularly preferred, the wavelength used for activating the photosensitizer, preferably one or more of the above-described phenothiazines, preferably, methylene blue and/or new methylene blue and/or toluidine blue, most preferred methylene blue, is at or around 590 nm such as from about 550 nm to about 630 nm, preferably about 570 to about 610 nm, more preferably about 585 nm to about 595 nm, most preferred 590 nm. Preferably, such light is emitted by an LED device, preferably a handheld device, which, in certain preferred embodiments of the invention can emit non-coherent light.

In other preferred embodiments of the invention, the invention makes use of the light absorption of phenothiazine photosensitizers as outlined herein in the near infrared region. In certain embodiments of this type, the photosensitizer-activating light is in the near infrared region of the electromagnetic spectrum, preferably from about 780 nm to about 820 nm, more preferably around or at 810 nm such as from about 800 nm to about 820 nm, most preferred 810 nm. Near infrared light has the advantage that it is absorbed to a lesser extend by body components so that it can reach into deeper into the irradiated regions as defined above. The fact of the wavelength dependency of light scattering which is higher for longer wavelengths further contributes to this effect, and at wavelengths of at or around 810 nm the scattering coefficient is higher than the absorption coefficient, and furthermore the scattering is a forward scattering further contributing to the higher depth of penetration. Such light preferably has a depth of penetration of about 5 to 10 cm which makes it particularly suitable for irradiation from the outside of the body.

In the context of irradiation in the near infrared region of the electromagnetic spectrum, preferably from about 780 nm to about 820 nm, more preferably around or at 810 nm such as from about 800 nm to about 820 nm, most preferred about 810 nm, the present invention also provides a photodynamic treatment, particularly for treatment of viral infections of the respiratory tract, more preferably for treatment and/or prevention of the viral infections as described above, by application of a photosensitizer such as a phenothiazine photosensitizer according to the definition as outlined above, more preferably selected from methylene blue, new methylene blue and toluidine blue, to a desired region, especially to a region of the respiratory tract (such as those as described above) of the subject to be treated, preferably a human, and transcutaneous irradiation of said region. In some embodiments of this aspects of the invention, the region of treatment also includes application of the photosensitizer and transcutaneous irradiation to one or both, preferably both, maxillary sinuses, and/or one or both, preferably both, paranasal sinuses, and/or one or both, preferably both, frontal sinuses. Besides the viral infections as outlined herein-above, such treatment will be also applicable to any infections, such as bacterial and/or viral and/or fungal, infections of the sinuses, preferably the above-mentioned sinuses.

Depending on the particular photosensitizer, it may be desired for efficient energy transfer to select a wavelength or wavelength region, respectively, at or at least near the absorption maximum of the photosensitizer. “Near the absorption maximum” in the context of the invention typically means a range around the absorption maximum of from about −50 nm to about +50 nm, preferably from about −20 nm to about +20 nm, more preferably from about −10 nm to about +10 nm of the absorption maximum. The absorption characteristics, including an absorption maximum of the photosensitizer, preferably of a phenothiazine derivative as described herein, is typically in the visible to near infrared range of the light spectrum, will depend on the biological setting, i.e. in which the photosensitizer is present as well as whether the subject is irradiated from outside of the body or within the body, and will also depend on and thus can be tuned by the components of a composition in which the photosensitizer(s) is present for application.

In other embodiments of the invention, the wavelength used for activating the photosensitizer is selected from regions of the, typically visible, absorption spectrum being more remote from an absorption maximum of the respective photosensitizer. In this context of the invention, the term “remote from an absorption maximum” of a photosensitizer for use in the invention refers to a wavelength.

The duration and number of times of irradiation of the at least part of the respiratory tract of the subject to be treated according to the invention varies according the specific photosensitizer, the wavelength or wavelength region of the light and on whether irradiation of the respiratory tract (or part(s) thereof) is carried from the outside or inside of the body of the subject to be treated. Typically, the irradiation is carried out, for example when irradiation is carried out from the outside of the subject's body, one or more times, such as 1, 2, 3, 4, 5, 6, 7, 8, 9, 10 times or more in one or more sessions at a day of treatment. The treatment can be repeated for two or more days, wherein the days of treatment can be uninterrupted or interrupted by one or more days where no treatment takes place. Preferably, the duration of the irradiation per irradiation incidence (i.e. one or more times as outlined above) is from about 1 min to about 20 min, more preferably from about 2 to about 20 min, particularly preferred from about 3 to about 8 min.

In preferred embodiments of the invention the at least part of the respiratory tract is irradiated, e.g. when irradiation is carried out from outside of the subject, with an energy density of 50 to 150 J/cm², preferably 80 to 120 J/cm, more preferably 90 to 110 J/cm, most preferably 100 J/cm². Preferred emitted light intensities, especially in the case of irradiation with non-coherent light, preferably by LED devices, more preferably handheld LED devices, such as broadband LED devices or LED devices emitting light at about or around about 590 nm (as outlined above) range from about 6000 Lux to about 90000 Lux, preferably from about 10000 Lux to about 70000 Lux, most preferred from about 40000 Lux to about 60000 Lux, such as, particularly preferred about 50000 Lux.

In general, the treatment of the invention is directed against any viruses causing a respiratory infection. Preferred viruses targeted by the inventive treatment are coronaviruses, more preferably coronaviruses causing severe respiratory conditions. Preferably, the inventive treatment is directed against SARS-CoV, MERS-CoV, SARS-CoV 2, HCoV-HKU1, HCoV-OC43, HCoV-NL63, HCoV-229E and/or bovine coronaviruses (bCoVs), with SARS-CoV-2 being the most preferred targeted virus which causes the COVID-19 disease.

In particular as regards coronaviruses such as those as outlined above, especially SARS-CoV-2, the inventive treatment is preferably carried out at an early stage of the infection. In such preferred embodiments, the term “early stage of the infection” means that the infecting virus has infected the subject, preferably a human subject, such that it has entered the respiratory tract, in particular the upper parts of the respiratory tract such as mouth, nose and/or pharyngeal part, but has not reached or has not reached in substantial extent, respectively, the lower parts of the respiratory tract, in particular the bronchia. In this context, especially with respect to coronaviruses such as SARS-Co-V, MERS and, particularly preferred, SARS-CoV-2, the viral infection has not resulted in a viral pneumonia. Thus, the inventive treatment is preferably used for preventing a viral pneumonia due to infection by a coronavirus, preferably due to an infection by SARS-CoV, MERS, or SARS-CoV-2. In other embodiments of the invention, the inventive treatment can also be used for prevention and/or treatment of a viral respiratory infection by applying the therapy of the invention to a subject which is suspected to be infected by a virus causing a respiratory infection or disease state, respectively. For example, especially in the case of viruses causing severe respiratory condition such as bronchitis and/or pneumonia, such as SARS-CoV, MERS and, particularly preferred in the context of the invention, SARS-CoV-2, the subject can be treated at time point after, preferably shortly after such as about 15 min, about 30 min, about 1 h, about 2 h, about 3 h, about 4 h, about 5 h, about 6 h, about 7 h, about 8 h, about 9 h, about 10 h, about 11 h, about 12 h or about 24 hours, the subject has gained information and/or has become aware and/or has been made aware of that the subject had close contact such as contact within a distance of below about 3 m, preferably below about 2 m, more preferably below about 1.5 m, and typically for a sufficient amount of time such as at least about 30 s, preferably at least about 1 min, more preferably at least about 2 min, with a subject having a viral respiratory infection such as an infection by SARS-CoV, MERS and/or, particularly preferred, SARS-CoV-2.

The present invention is also directed to a medical kit comprising the pharmaceutical device as described and defined herein and a light source emitting light comprising or having a wavelength causing the photosensitizer to be transferred to an active state. Preferred embodiments of such light sources have been elaborated above.

The present invention is also directed to a method for prevention and/or treatment of a viral respiratory infection such as preferably by coronaviruses, preferably those as outlined herein before, comprising the steps of (i) administering at least one photosensitizer to at least a part of the respiratory tract of the subject and (ii) irradiating said at least part of the respiratory tract of the subject with light having a wavelength causing the photosensitizer to be transferred in to an activated state.

Preferred embodiments of photosensitizers and compositions containing same, administration regimens, light sources and light emitting devices, wavelengths, duration and times of irradiation for use in the inventive treatment method have already described herein above.

Further preferred details and embodiments the invention, in particular treatment conditions as well as devices, photosensitizers, light sources etc. have been described herein-before and will further be exemplified in the examples.

### BRIEF DESCRIPTION OF THE DRAWINGS

FIG. 1 shows a table illustrating the results of the experiments according to Example 1.

FIG. 2 shows a table illustrating the results of the experiments according to Example 3 (VC: virus control; MV: mean value; SD: standard deviation; RF: reduction factor; n.a.: not applicable; n.d.: not done; the results of the experiments according to the last two lines of the table were obtained after immediate wrapping of the experimental plate with aluminium foil and storage in the dark (before freezing).

### DETAILED DESCRIPTION OF THE INVENTION

The present invention is further illustrated by the following non-limiting examples:

### EXAMPLES

### Example 1: Inactivation of Bovine Coronavirus in Infected Cells In Vitro

Materials

Laser device: Photolase 810 Diode Laser of the applicant: max. Laser Output Power: 7 Watt; Laser; Wavelength: 810 nm/7 Watt)
The laser device contains a handpiece allowing irradiation without loss of performance in a distance up to 105 cm with a light cone laser of approximately 1 cm diameter. In some experiments, the laser was fixed on a tripod in a distance of 30 cm to ensure the same conditions for all tests.

Irradiation Conditions
Power density: 0.3 Watt
Irradiation Intensity: 100 Joule/cm²
Amount of sensitizer/well of 48-well plate: 0.1%
0.01%
0.001%
0.0001%
Test temperature (irradiation): 20° C.±2° C.
Incubation temperature (titration): 37° C.±1° C.

Photosensitizer
Photolase Blueviolett Sensitizer 810: Methylene blue stock solution containing 1.07% (w/v) methylene blue.

Cells and Viruses
BCoV strain L9 was obtained from Dr. G. Zimmer, Institute of Virology at the Tierärztliche Hochschule Hannover, Germany.
U373 cells (passage 14) were obtained from Dr. G. Zimmer, Institute of Virology at the Tierärztliche Hochschule Hannover, Germany.

Media and Auxiliary Reagents
Eagle's Minimum Essential Medium with Earle's BSS (EMEM, Biozym Scientific GmbH, catalogue no. 880121)
Fetal calf serum (FCS, Sigma-Aldrich, article no. F 7524)
Aqua bidest. (SG ultrapure water system, type Ultra Clear, serial no. 86996-1)
PBS (Invitrogen, article no. 18912-014)
Trypsin-EDTA solution
Penicillin/streptomycin (Sigma-Aldrich, article no. P-0781)
Trypsin stock solution [2.5 mg/ml]

Methods
To analyse the efficacy of the inventive photoactivated treatment for inactivation of bovine coronavirus, U373 cells were cultivated in 48-well plates and infected with BCoV before irradiation treatment. The following general steps were carried out:
(1) (Sub)culturing of U373 cells in 48-well plates
(2) 150 μl cell suspension+500 μl medium (EMEM 10% FKS)
(3) Infection of the U373 cells with 200 μl of virus-medium-mixture
(4) Pre-treatment and irradiation of the BCoV-infected cells

Preparation of Test Virus Suspension
For preparation of test virus suspension, U373 cells were cultivated in a 175 cm2 flask with in EMEM supplemented with L-glutamine, non-essential amino acids and sodium pyruvate and 10% fetal calf serum. Before virus infection, cells were washed two times with phosphate buffered saline (PBS), incubated for 3 h with EMEM without FCS whereafter the cells were washed once with EMEM supplemented with trypsin. For virus production, BCoV strain L9 was added to the prepared monolayer. After an incubation period of 24 to 48 hours (cells showed a constant cytopathic effect), cells were lysed by a rapid freeze/thaw cycle.
Cellular debris was removed by low speed centrifugation. After aliquotation of the supernatant, test virus suspension was stored at −80° C.

Preparation of U373 Cells for Irradiation Treatment
U373 cells of a 175 cm2 cell culture flask were detached enzymatically with Trypsin-EDTA solution and taken up in a total of
60 ml of EMEM medium with 10% fetal calf serum. 150 μl each of this cell suspension were transferred into six wells of a 48-well plate with a final volume of 650 μl by adding 500 μl EMEM/10% FCS.
48-well plates were selected, since the diameter of one well corresponds to the diameter of the laser cone (light cone laser: approx. 1 cm diameter; well of 48-well plate: 1.04 cm diameter), which ensures that the cells are treated evenly throughout the entire well during radiation.
After three days of cultivation at 37° C. and 5% CO2, cells were washed two times with EMEM without FCS (2×200 μl per well) and incubated for further 3 h at 37° C. and 5% CO2. For virus infection, medium was removed from the individual wells and replaced by 200 μl virus-medium-mixture (500 μl BCoV virus suspension were mixed with 30 ml EMEM without FCS/with penicillin/streptomycin, and trypsin). After an incubation period of 20 to 24 hours BCoV-infected cells were used for irradiation treatment.

Preparation of Photosensitizer
The methylene blue photosensitzier Photolase® Blueviolett Sensitizer 810 was used in the following concentrations based on the content of 1.07% (w/v) methylene blue in the undiluted sensitizer:
(a) 0.1% (w/v) (end concentration in 200 μl/well)
(b) 0.01% (w/v) (end concentration in 200 μl/well)
(c) 0.001% (w/v) (end concentration in 200 μl/well)
(d) 0.0001% (w/v) (end concentration in 200 μl/well)

Irradiation Procedure
For photodynamic inactivation of the bovine coronavirus (BCoV) first the undiluted or appropriately diluted (see above) photosensitizer solution (18.69 μl per well) was added to the BCoV-infected cells for 1 minute. Thereafter, irradiation with the above laser device was performed with an intensity of 100 joule/cm² for 5.46 min under constant power of 0.3 watt/cm2 and artificial laboratory light.
After treatment, plates were stored at −80° C., successively.

The following Table 1 shows the irradiation conditions used in the present Example 1.

TABLE 1
Irradiation conditions according to Example 1

| | | Conc. of | Irradiation | Incubation |
|---|---|---|---|---|
| BCoV | Power | sensitizer in | time at 100 | time under |
| infected | density | 200 μl/well | J/cm² | artificial light |
| U373 cells | [watt/cm²] | [(w/v)] | [min] | [min] |
| non-treated | — | — | — | — |
| (virus control) | | | | |
| treated | 0.3 | 0.1 | 5.46 | 5.46 |
| treated | 0.3 | 0.01 | 5.46 | 5.46 |
| treated | 0.3 | 0.001 | 5.46 | 5.46 |
| treated | 0.3 | 0.0001 | 5.46 | 5.46 |
| treated | — | 0.1 | — | 10.0 |
| treated | — | 0.01 | — | 10.0 |
| treated | — | 0.001 | — | 10.0 |
| treated | — | 0.0001 | — | 10.0 |
| treated | — | 0.01 | — | Immediate |
| | | | | transfer into |
| | | | | the dark |
| treated | 0.3 | — | 5.46 | 5.46 |

Recovery of the residual virus and determination of infectivity For recovery of residual virus from the infected and treated cells, plates were subjected to a rapid freeze/thawing procedure. This was followed by mixing of cell suspension in each well by pipetting up and down 10 times to re-suspend the virus. Then, series of ten-fold dilutions of the suspensions were prepared in ice-cold maintenance medium, respectively. Finally, 100 μl of each dilution were placed in eight wells of a sterile polystyrene flat-bottomed plate with a preformed U373 monolayer Before the addition of the respective dilution, cells were washed twice with EMEM, and incubated for 3 hours 100 μl EMEM supplemented with trypsin. The cells were incubated at 37° C. in a CO2-atmosphere (5.0% CO₂ content). After six days of incubation, cultures were observed for cytopathic effects. The infectious dose (TCID50) was calculated according to the method of Spearman (1908) Brit. J. Psychol. 2, 227-242, and Kärber (1931) Arch. Exp. Path. Pharmak.; 162, 480-487.

Controls
(i) Virus control (VC)
Virus recovery was performed from non-treated infected U373-cells as described under 3.5. The mean virus titre was used as reference for calculation of the reduction factor.
(ii) Irradiation without photosensitizer
In addition, virus recovery was performed from infected and radiated U373-cells without using of the Photolase sensitizer as
(iii) Treatment with photosensitizer (without irradiation)
Another virus recovery was performed from infected U373-cells, treated with the Photolase sensitizer for 10 min under artificial laboratory light only (without radiation) as described in 3.5, whereby one Plate was wrapped up with aluminium foil immediately after addition of the dye (without radiation or light exposing) and stored and frozen in the dark until titration of residual virus.
(iv) Cell culture control

Furthermore, a cell control (only addition of medium) was incorporated.

Calculation of Effectiveness

The virucidal effectiveness of the inventive treatment was evaluated by calculating the decrease in titre of the treated and radiated culture in comparison with the control titration of the culture without treatment (VC). The difference is given as reduction factor (RF).

Based on the EN 14476, the present photodynamic therapy system has a virucidal efficacy if the titre is reduced at least by 4 log 10 steps after an irradiation treatment (EN 14476:2013+A2:2019: Chemical disinfectants and antiseptics—Quantitative suspension test for the evaluation of virucidal activity of chemicals disinfectants and antiseptics in human medicine test—Test method and requirements (phase 2, step 1)). This corresponds to an inactivation of ≥99.99%.

Results

The effectiveness of the treatment of the infected cells according to the invention was determined after the irradiation treatment of three BCoV-infected U373 cell cultures of a 48-well plate (corresponds to three wells), respectively. Results of examination are shown in the following Table 2.

TABLE 2
Results of treatment of BCoV-infected cells according to
Example1

| | | Concentration of | |
|---|---|---|---|
| BCoV infected | Power density | photosensitizer | Irradiation time at |
| U373 cells | [W/cm²] | in 200 μl/well [%] | 100 J/cm2 |
| treated | 0.3 | 0.1 | 5.46 |
| treated | 0.3 | 0.01 | 5.46 |
| Treated | 0.3 | 0.001 | 5.46 |
| treated | 0.3 | 0.0001 | 5.46 |

After adding the photosensitizer to the culture of BCoV-infected cells and irradiation with 810 nm at an energy density of 100 joule/cm² and under a constant power density of 0.3 watt/cm² no residual virus was found using 0.1% (w/v) to 0.001% (w/v) of the photosensitizer The reduction factor (RF) varied from 3.08 (with 0.1% of the dye), to 4.08 (0.01%), and 5.08 (0.001%). When using 0.0001% of the photosensitizer only minimal residual virus could still be detected in one sample. However, the mean reduction factor was each ≥6.00 log₁₀ steps, since no cytotoxicity (CT) could be observed in these samples. This corresponded to a virus inactivation of ≥99.999%.

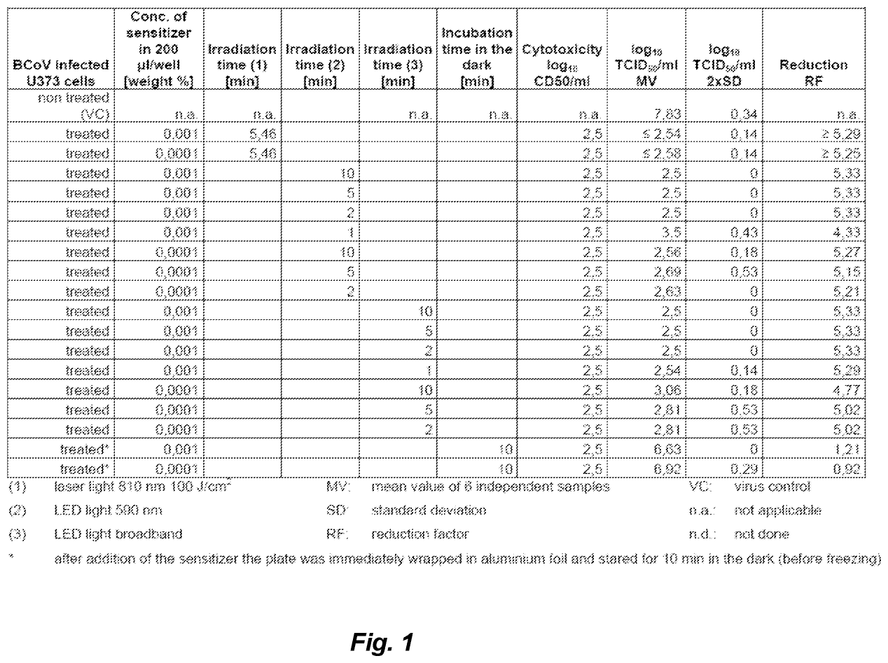

In contrast, in tests of BCoV-infected cells that were treated with 0.01% of photosensitizer (without radiation) and immediately wrapped in aluminium foil after addition of the sensitizer, and stored and frozen in the dark until titration of residual virus), substantial amounts of residual virus and no effectiveness could be detected (RF=0.38±0.64) (FIG. 1).

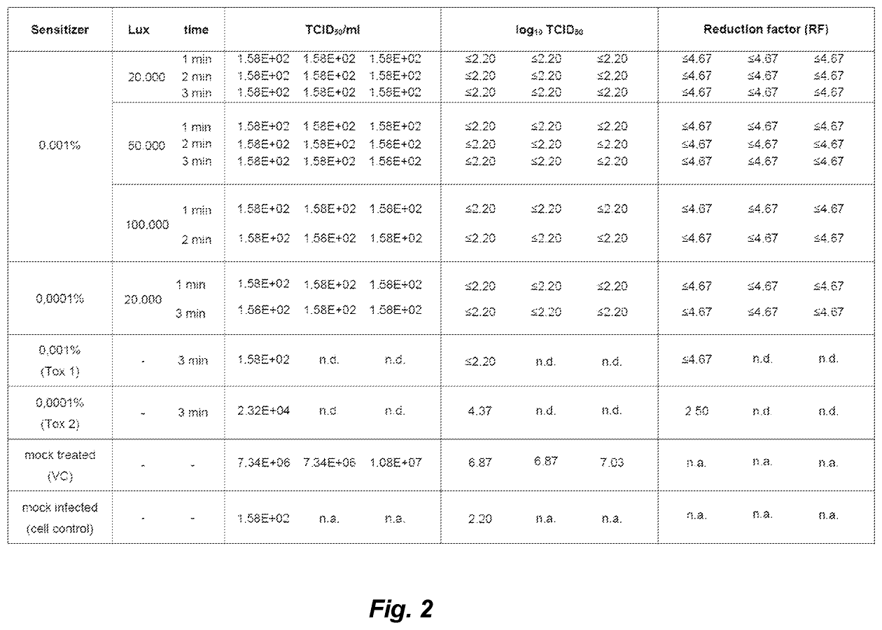

Test samples that were only irradiated and not treated with the sensitizer beforehand showed also no effectiveness (RF=0.08±0.64) (FIG. 2).

The present Example shows that the inventive treatment of cells infected with a high dose of bovine coronavirus leads to an effective virus inhibition using a composition containing the photosensitizer at concentrations of as low as 0.0001% (w/v).

### Example 2: Inactivation of Bovine Coronavirus in Infected Cells In Vitro Using Non-Coherent LED Light

In a further set of experiments, the U737 cells were treated under the conditions as shown in the table of FIG. 1 using irradiation with either a monochromatic LED device at 590 nm or a broadband LED device (non-coherent light in each case). Light intensity was 10000 Lux in both cases.

Surprisingly, broadband LED light was successfully applied for photosensitizer activation leading to reduction of residual virus by 3 log₁₀ steps after 2 min of irradiation.

### Example 3 Inactivation of SARS-CoV-2 in Infected Cells In Vitro

Virus and Cells
The SARS-CoV-2/Germany strain was derived from a patient isolate.
The Vero E6 cells were obtained from University Bern, Switzerland.
The cells were inspected regularly for morphological alterations and for contamination by mycoplasmas. No morphological alterations of cells and no contamination by mycoplasmas could be detected.

Light Source and Irradiation Conditions
Light source
Ledlenser P6 (Ledlenser GmbH & Co. KG, Solingen, Germany)

Irradiation Conditions
Illuminance: 20,000 lux, 50,000 lux or 100,000 lux
Concentration of sensitizer/well of 24-well plate: 0.001% (w/v) or 0.0001% (w/v)
Test temperature (irradiation): 20° C.±2° C.
Incubation time(s): 1, 2 or 3 minutes
Incubation temperature (titration): 37° C.±1° C.

Other Materials

Culture Medium and Reagents
Dulbecco's Modified Eagle Medium (DMEM, Thermo Fisher, catalogue no. 11965092)
Fetal Bovine Serum (FBS, Thermo Fisher, article no. 10270106)
Penicillin-Streptomycin (P/S, Thermo Fisher, catalogue no. 15140122)
Non-Essential Amino Acids Solution (100×) (NEAAs, Thermo Fisher, catalogue no. 11140035)
L-Glutamine (200 mM) (L-Glut, Thermo Fisher, catalogue no. 25030024)
Trypsin-EDTA (0.5%), no phenol red (Thermo Fisher, catalogue no. 15400054
BSA (Sigma-Aldrich-Chemie GmbH, article no. CA-2153)
sheep erythrocytes (Fiebig Nährstofftechnik)
Crystal Violet (Sigma-Aldrich-Chemie GmbH, article no. C0775)

Apparatus, glassware and small items of equipment
CO₂ incubator
Agitator (Vortex Genie Mixer)
Microscope, inverse
Centrifuge, water bath
Adjustable and fixed-volume pipettes
24-well plates and 96-well microtitre plates
Cell culture flasks and sealed reaction test tubes.

Methods

For analyzing the efficacy of the photosensitizer activation to inactivate the humane coronavirus, Vero E6 cells were cultivated in24-well plates and infected with SARS-CoV-2 before irradiation treatment. The following short flowchart provides an overview of the process:

(Sub)culturing of Vero cells in 24-well plates:
1×105 cells/well—1 ml medium/well—1 well/plate

(Day 1)
Infection of the Vero cells with SARS-CoV-2 (MOI 3):
500 μL virus suspension/well for 1 hour—wash with PBS (1×)—add 1 ml medium/well

(Day 2)
Pre-treatment/irradiation of the SARS-CoV-2-infected cells:
Pretreatment with 0.001% or 0.0001% Sensitizer and irradiation for 1, 2 and 3 minutes (immediately after treatment: plates were wrapped in aluminium foil) Harvest: Remove supernatant, wash 1× with PBS, add 500 μL PBS and freeze

(Day 3)
Virus recovery (3× freeze/thaw procedure) and virus titration

Preparation of Test Virus Suspension

For virus production, 2×106 Vero E6 cells were cultivated in a 75 cm2 flask in DMEM supplemented with 1% L-Glut, NEAAs, and P/S and 10% FBS. One day after seeding, medium was changed to 10 ml fresh DMEM inoculated with 100 μl of SARSCoV-2/Germany virus suspension. The supernatant was harvested after 3 days at 37° C. by centrifugation at 1,500 rpm for 5 min to remove cell debris. The supernatant was aliquoted and stored at −80° C. Viral titres were determined by plaque assay and endpoint dilution.

Preparation of Vero Cells for Irradiation Treatment

Vero E6 cells of a cell culture flask were detached enzymatically with Trypsin-EDTA solution. 1×105 cells were transferred into one well (B3) of a 24-well plate with a final volume of 1,000 μl cell culture medium. After one day of cultivation at 37° C. and 5% CO2, medium was removed from the individual wells and cells were infected with SARS-CoV-2 (500 μL virus suspension per well 2×200 μl per well, MOI 3). After 1 h of incubation at 37° C., inoculum was removed, cells were washed once with PBS and cultivated in 1 ml culture medium for further 20 to 24 h at 37° C. and 5% CO2. After that, SARS-CoV-2-infected cells were used for irradiation treatment.

Preparation of Sensitizer

The following methylene blue solutions were prepared using a stock solution containing 1.07% (w/v):
a) 0.001% (w/v) (end concentration in 1,000 μl/well)
b) 0.0001% (w/v) (end concentration in 1,000 μl/well)
For the 0.001% (w/v) methylene blue concentration 1 μl of the undiluted sensitizer was added to 1000 μl medium per well (plate swayed). For the further concentration of 0.0001% (w/v) the photosensitizer was diluted 1:10 in Aqua dest. and 1 μl of the dilution were added to 1,000 μl medium.

Manual Irradiation Procedure with Different Photodynamic Systems

For photodynamic inactivation of human coronavirus (SARS-CoV-2), first the appropriately diluted methylene blue solution (1 μl per well) was added to the SARS-CoV-2-infected cells. Immediately thereafter, irradiation with the Ledlenser P6 was performed with a light intensity of 20,000 Ix, 50,000 Ix, and 100,000 Ix, respectively. The single well of each plate was treated separately.

TABLE 3
Photosensitizer and irradiation conditions for treatment
samples and controls

| | | Irradiation | Irradiation | Irradiation |
|---|---|---|---|---|
| | Conc. of | time | time | time |
| SARS-CoV-2 | sensitizer in | at irradiation | at irradiation | at irradiation |
| infected | 1000 μl/well | with 100,000 | with 100,000 | with 100,000 |
| Vero E6 cells | [% (w/v)] | lx [min] | lx [min] | lx [min] |
| untreated | — | — | — | — |
| (virus control) | | | | |
| treated (Tox 1) | 0.001 | — | — | — |
| treated (Tox 2) | 0.0001 | — | — | |
| treated | 0.001 | 1 | 1 | 1 |
| treated | 0.001 | 2 | 2 | 2 |
| treated | 0.001 | 3 | 3 | 3 |
| treated | 0.0001 | 1 | — | — |
| treated | 0.0001 | — | — | — |
| treated | 0.0001 | 3 | — | — |

After treatment, the entire plate was immediately wrapped in aluminum foil for maximum 1 min. Afterwards supernatant was removed, cells were washed once with 1,000 μl PBS, overlaid with 500 μL PBS and stored at −80° C.

Recovery of the residual virus and determination of infectivity For recovery of residual virus from the infected and treated cells, plates were subjected to three freeze/thawing procedures. This was followed by mixing of cell suspension in each well by pipetting up and down 15 times to re-suspend the virus. After that, 22 μl of the virus-disinfectant solution was immediately added to the first row of Vero E6 cells (seeded at 1×104 cells/well in a 96 well plate one day prior the examination), followed by a serial endpoint dilution titration. After 3 days of incubation at 37° C. in a CO2-atmosphere (5.0% CO2-content) cultures were observed for cytopathic effects by crystal violet staining. The infectious dose (log 10 TCID50/ml) was calculated according to the method of Spearman (1) and Kärber (2).

Controls
(a) Virus control (VC)
Virus recovery was performed from non-treated SARS-CoV-2-infected Vero E6 cells (no sensitizer and no irradiation) as described above. The mean virus titre was used as reference for calculation of the reduction factor (RF).
(b) Treatment with methylene blue solution without irradiation
Another virus recovery was performed from infected Vero E6 cells, treated with the indicated methylene blue solution for 3 min in darkness. After treatment, culture plate was wrapped up with aluminum (without exposure to light), stored for a maximum of 1 min in the dark before harvest (remove supernatant, washing with PBS (1×), adding 500 μL PBS and frozen until virus recovery and titration of the residual virus) was performed as described above.
(c) Cell culture control
Furthermore, a cell control (only addition of medium) was incorporated.

Calculation of Effectiveness

The virucidal effectiveness of the methylene blue treatment and the photodynamic inactivation properties with ambient light was evaluated by calculating the decrease in titre of the treated and radiated culture in comparison with the control titration of the approaches without treatment (VC). The difference is given as reduction factor (RF).

Results

The effectiveness of the photodynamic systems was determined in triplicate after the irradiation treatment of SARS-CoV-2-infected Vero E6 cells (only one well of a 24-well plate per concentration of the sensitizer and irradiation condition). The results of the experiments examination are shown in FIG. 2.

No residual virus was found with 0.001% or 0.0001% of photosensitizer and subsequent irradiation for just 1 minute with an irradiation intensity as low as 20,000 Ix and an average initial virus titre of 6.92 log 10 TCID50/ml. The mean reduction factor (RF) was 4.67 or more for both photosensitizer concentrations (0.001 or 0.0001, respectively), which corresponds to an inactivation of the SARS-CoV-2 of 99.99%.

### CONCLUSION

A surprisingly high reduction in virus titer was obtained using 0.001% (w/v) of the sensitizer and irradiation with broadband light after an incubation time of only 1 min.

### Example 4 Treatment of a Human Subject Suffering from COVID-19

Patient: male Caucasian, age 58
Condition of patient before treatment: positive PCR SARS-CoV-2, cycle threshold (CT)=19; dry cough, headache, body temperature 38.2° C., medium general condition
Treatment: for 3 days every 6 hours: 3 μg methylene blue (0.00107 (w/v) in aqueous solution) per treatment of both nostril and throat by administration using a nasal spray device into each nostril and into the mouth//throat (each pump-spray action of the device administers 1 μg of methylene blue in a volume of 0.1 ml; irradiation using handheld LED broadband handheld device (WUBEN® L50 obtained from Shenzhen Shengqi Lighting Technology Co., Ltd., Shenzhen, China) employing an Osram P9 LED emitting warm white light, 1200 Im) for a duration of 2 min in each nostril and 2 min in throat at ca. 6000 Lux directly after the application of the photosensitizer.
Condition of patient after days of treatment: no cough, no headache, normalized body temperature, normal general condition; PCR SARS-CoV-2, CT=35

This Example shows that treatment of a patient showing symptoms of early to middle conditions of SARS-CoV-2 infections who, given an age of above 55, is in general danger of developing more severe COVID-19 symptoms, substantially recovers through a treatment lasting only 3 days by application of photosensitizer and irradiation using broadband LED device every 6 hours. Moreover, the resulting PCR CT of 35 after 3 days of this treatment indicates that the patient is comparatively unlikely to spread SARS-CoV-2 according to Reference (3) noted below.

### REFERENCES

(1) Spearman, C.: The method of ‘right or wrong cases’ (constant stimuli) without Gauss's formulae. Brit J Psychol; 2, 1908, 227-242
(2) Kärber, G.: Beitrag zur kollektiven Behandlung pharmakologischer Reihenversuche. Arch Exp Path Pharmak; 162, 1931, 480-487
(3) Singanayagam, A, et al.: Duration of infectiousness and correlation with RT-PCR cycle threshold values in cases of COVID-19, England, January to May 2020. Euro Surveill. 2020; 25 (32):pii=2001483. https://doi.org/10.2807/1560-7917.ES.2020.25.32.2001483

1-16. (canceled)

17. A pharmaceutical device comprising a nebulizer and a container containing at least one photosensitizer, optionally in combination with at least one pharmaceutically acceptable excipient and/or diluent.

18. The pharmaceutical device of claim 17 wherein the photosensitizer comprises a photoactivatable phenothiazine derivative, preferably a photoactivatable phenothiazine dye, more preferably a phenothiazine derivative having a structure of the following formula (I) 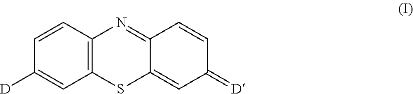
wherein
D and D′ each are an electron donor group, and wherein the positions C-1, C-2, C-4, C-6, C-8 and/or C-9 of the phenothiazine ring system may be substituted by a substituent preferably selected from substituted or unsubstituted lower alkyl, preferably C₁₋₃-alkyl, particularly preferred methyl, or lower alkenyl, preferably C₂₋₃-alkenyl, amino, halo, and cyano, and preferably, D and D′ are each independently a NRR′ group with R and R′ independently selected from H, substituted or unsubstituted lower alkyl, and lower alkenyl.

19. The pharmaceutical device of claim 18 wherein the photosensitizer is present in sterilized water or in a sterilized aqueous solution.

20. The pharmaceutical device of claim 18 wherein the container contains the photoactivatable phenothiazine derivative at a concentration of from 0.00001 (v/v) to 0.1% (v/v), preferably from 0.0001% (v/v) to 0.001% (v/v).

21. The pharmaceutical device of claim 18, further comprising a light source emitting light comprising or having a wavelength causing the photosensitizer to be transferred to an activated state.

22. (canceled)

23. The pharmaceutical device of claim 21 wherein the light source is an LED device.

24. The pharmaceutical device of claim 23 wherein the light source is a broadband LED device.

25. The pharmaceutical device of claim 23 wherein the LED device is a laser LED device.

26. The pharmaceutical device of claim 25 wherein the laser LED device emits light having a wavelength in the range of from 550 nm to 630 nm, preferably 570 to 610 nm, more preferably 585 nm to 595 nm, most preferred the light has a wavelength of 590 nm.

27. The pharmaceutical device of claim 25 wherein the laser LED device emits light having a wavelength in the range of from 780 nm to 820 nm, preferably from about 800 nm to about 820 nm, most preferred the light has a wavelength of 810 nm.

28. The pharmaceutical device of claim 23 wherein the LED device emits in-coherent monochromatic light having a wavelength in the range of from 550 nm to 630 nm, preferably 570 to 610 nm, more preferably 585 nm to 595 nm, most preferred the light has a wavelength of 590 nm.

29. The pharmaceutical device of claim 23 wherein the LED device emits in-coherent monochromatic light having a wavelength in the range of from 780 nm to 820 nm, preferably from about 800 nm to about 820 nm, most preferred the light has a wavelength of 810 nm.

30. The pharmaceutical device of claim 21 wherein the light source comprises a handheld torch-type broadband LED device containing a light-guiding element and/or positioning element removably attached to an end of the torch-type LED device where emitted light exits the LED device.

31. The pharmaceutical device of claim 18, wherein R and R′ are independently selected from H, C₁₋₃-alkyl, most preferably methyl, and C₂₋₃-alkenyl.

32. A method for prevention and/or treatment of a viral respiratory infection in a subject, comprising the steps of:
(i) administering at least one photosensitizer to at least a part of the respiratory tract of the subject and
(ii) irradiating the at least a part of the respiratory tract of the subject with light having a wavelength causing the photosensitizer to be activated.

33. The method of claim 32 wherein the photosensitizer is applied intra-nasally and/or intra-orally and/or pharyngeally.

34. The method of claim 32 wherein the step of irradiating the at least a part of the respiratory tract of a subject comprises irradiating from outside of the subject.

35. The method of claim 32 wherein the light is provided by a light emitting diode (LED) device, preferably a handheld LED device.

36. The method of claim 32 wherein the light is provided by a laser LED device, preferably a handheld laser LED device, or a non-coherent light emitting LED device, preferably a broadband LED device.

37. The method of claim 32 wherein the at least a part of the respiratory tract is irradiated one or more times for a period of from 2 to 20 min, preferably from 2 to 10 min, more preferably from 3 to 8 min.

38. The method of claim 32 wherein the at least part of the respiratory tract is irradiated from outside of the subject with an energy density of 50 to 150 J/cm² preferably 80 to 120 J/cm, more preferably 90 to 110 J/cm, most preferably 100 J/cm².

39. The method of claim 32 wherein the photosensitizer is a photoactivatable phenothiazine derivative, preferably a photoactivatable phenothiazine dye, more preferably a phenothiazine derivative having a structure of the following formula (I) 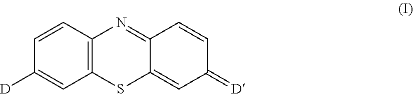
wherein the positions C-1, C-2, C-4, C-6, C-8 and/or C-9 of the phenothiazine ring system may be substituted by a substituent preferably selected from substituted or unsubstituted lower alkyl, preferably C₁₋₃-alkyl, particularly preferred methyl, or lower alkenyl, preferably C₂₋₃-alkenyl, amino, halo, and cyano; and
D and D′ are each independently a NRR′ an electron donor group group with R and R′ being preferably independently selected from H, substituted or unsubstituted lower alkyl, preferably C₁₋₃-alkyl, particularly preferred methyl, and lower alkenyl, preferably C₂₋₃-alkenyl.

40. The method of claim 39 wherein the phenothiazine dye is selected from the group consisting of methylene blue, new methylene blue, toluidine blue and mixtures thereof.

41. The method of claim 39 wherein the photoactivatable phenothiazine derivative is applied as a composition containing from 0.00001 (v/v) to 0.1% (v/v), preferably from 0.0001% (v/v) to 0.001% (v/v), of said photoactivatable phenothiazine derivative.

42. The method of claim 39 wherein the treatment comprises irradiating at least a part of the respiratory tract of a subject from outside of the subject with light comprising or having a wavelength selected from 500 nm to 820 nm of the electromagnetic spectrum.

43. The method of claim 42 wherein the light has a wavelength in the range of from 550 nm to 630 nm, preferably 570 to 610 nm, more preferably 585 nm to 595 nm, most preferred the light has a wavelength of 590 nm.

44. The method of claim 42 wherein the light has a wavelength in the range of from 780 nm to 820 nm, preferably from about 800 nm to about 820 nm, most preferred the light has a wavelength of 810 nm.

45. The method of claim 39 wherein the viral respiratory infection is caused by a coronavirus.

46. The method of claim 45 wherein the coronavirus is selected from the group consisting of SARS-CoV, MERS-CoV, SARS-CoV 2, HCoV-HKU1, HCoV-OC43, HCoV-NL63, HCoV-229E and bovine coronaviruses (bCoVs).

47. The method of claim 46 wherein the coronavirus is SARS-CoV 2.
